(19) United States
(12) Patent Application Publication
(10) Pub. No.: US 2024/0024637 A1
(43) Pub. Date: Jan. 25, 2024

(54) DETACHABLE GUIDE WIRE ASSEMBLY WITH VARIABLE DIAMETER

(71) Applicant: BARD PERIPHERAL VASCULAR, INC., Franklin Lakes, NJ (US)

(72) Inventor: Matt Casiraro, Tempe, AZ (US)

(21) Appl. No.: 18/038,726

(22) PCT Filed: Dec. 3, 2020
(86) PCT No.: PCT/US2020/063073

§ 371 Date: May 25, 2023

(51) Int. Cl. A61M 25/09

(52) U.S. Cl. CPC A61M 25/09; A61M 2025/09058

(57) ABSTRACT

A detachable guidewire assembly (10) is provided with multiple diameter compatibility. The assembly (10) includes a first guidewire (12) and second guidewire (14) a diameter less than that of the first guidewire. The second guidewire (14) is adapted for insertion and removal within and from the first catheter (12) via a receiver in form of an open lateral channel (16) or a longitudinal bore. A releasable coupling (18) is provided for selectively connecting the first guidewire (12) to the second guidewire (14). The releasable coupling may be formed by a magnetic coupling or a mechanical coupling. The second guidewire (14) may be equal to or greater in length than the first guidewire (12). In a coupled configuration, the distal tips of the first and second guidewires may align, but a proximal end portion of the second guidewire (14) may extend proximally of the first guidewire (12).

### BACKGROUND OF THE INVENTION

Balloon dilatation catheters are often used to treat lesions in vessels. Such catheters are typically guided to a treatment location in the vessel by a guidewire. Typically, the guidewire is a small diameter, highly flexible piece of a metal, such as stainless steel, sized to track easily through the vasculature from an access point and provide reliable guidance to the balloon catheter.

Guidewires of different diameters may be used during a given procedure, depending on the treatment location. For example, larger vessels in the “upper” vasculature (aortic/iliac/femoral) may use a larger diameter guidewire than smaller vessels in the peripheral vasculature, or “below the knee” (arterial popliteal/tibial/pedal). This variability dictates that the clinician stock a variety of different sizes of guidewire on hand, which can increase costs and complexity. When not done in situ, performing guidewire exchanges can also be time-consuming during an invasive procedure, where efficiency can be extremely important.

Past proposals for guidewire assemblies have been made, which contemplate connecting guidewires of different sizes into a single unit. However, the known proposals suffer from a lack of ease of use.

### SUMMARY OF THE INVENTION

An object of the disclosure is to provide a detachable guidewire assembly, which can be selectively adapted for use in different areas of the vasculature in an easy and efficient manner.

According to one aspect of the disclosure, a detachable guidewire assembly includes a first guidewire having a first diameter and including a lateral channel and a second guidewire having a second diameter less than the first diameter, such as to provide multiple diameter compatibility. The second guidewire is adapted for being inserted within and removed from the lateral channel. A releasable coupling is provided for selectively connecting the first guidewire to the second guidewire.

In one embodiment, the releasable coupling comprises a magnetic coupling. The magnetic coupling may be formed by a plurality of magnets arranged in pairs spaced along the first guidewire and the second guidewire. Alternatively, the releasable coupling may comprise a transverse pin.

In one embodiment, the lateral channel includes an open end having a dimension greater than the second diameter. The first diameter may be from approximately 0.035 inches to 0.039 inches and the second diameter is from approximately 0.014 to 0.018 inches. The first guidewire may comprise a C-shaped cross-section.

According to a further aspect of the disclosure, a detachable guidewire assembly having multiple diameter compatibility comprises a first guidewire having a first diameter, a second guidewire having a second diameter less than the first diameter, and a magnetic coupling for selectively connecting the first guidewire to the second guidewire.

In one embodiment, the first guidewire includes a receiver adapted for receiving the second guidewire. The receiver may comprise a bore or a lateral channel adapted for receiving the second guidewire. The lateral channel includes an open end formed by a surface of the first guidewire and having a dimension greater than the second diameter.

The magnetic coupling may be formed by a plurality of magnets arranged in pairs spaced along the first and second guidewire. The first diameter may be from approximately 0.035 inches to 0.039 inches and the second diameter may be from approximately 0.014 inches to 0.018 inches. The first guidewire may comprise a C-shaped cross-section.

According to another aspect of the disclosure, a detachable guidewire assembly includes a first guidewire having a receiver, a second guidewire; and a releasable coupling within the receiver for selectively connecting the first guidewire to the second guidewire.

In one embodiment, the releasable coupling comprises a magnetic coupling. The magnetic coupling may be formed by a plurality of magnets arranged in pairs spaced along the first guidewire and the second guidewire. The releasable coupling may comprise a transverse pin.

In one embodiment, the receiver includes an open end having a dimension greater than a diameter of the second guidewire. The first diameter of the first guidewire may be from approximately 0.035 inches to 0.039 inches and a second diameter of the second guidewire is from approximately 0.014 to 0.018 inches.

Still a further aspect of the disclosure relates to detachable guidewire assembly having multiple diameter compatibility. The assembly comprises a first guidewire including a receiver and a second guidewire adapted to be located in the receiver. A transverse pin is provided for selectively connecting the first guidewire to the second guidewire.

In one embodiment, the first guidewire includes a transverse passage for receiving the transverse pin so as to pass through a notch in the second guidewire when located in the receiver. The receiver may comprise a bore in the first guidewire. A first diameter of the first guidewire may be from approximately 0.035 inches to 0.039 inches and a second diameter of the second guidewire may be from approximately 0.014 to 0.018 inches.

Yet another aspect of the disclosure pertains to a method of performing a medical procedure in a vasculature. The method includes inserting into the vasculature a guidewire assembly formed of a first guidewire releasably coupled to a second guidewire having a smaller diameter than the first guidewire. After the inserting step, the method involves separating the first guidewire from the second guidewire.

In one embodiment, the separating step comprises breaking a magnetic coupling formed among the first guidewire and second guidewire. In this or another embodiment, the separating step comprises releasing a mechanical coupling formed between the first guidewire and second guidewire. The separating step may further include peeling away the second guidewire from a lateral channel in the first guidewire, or pulling or sliding the second guidewire from a bore in the first guidewire.

In any disclosed embodiment, the second guidewire may have a second length equal to or greater than a first length of the first guidewire.

Still a further aspect of the disclosure pertains to a detachable guidewire assembly. The assembly comprises a first guidewire including a receiver, the first guidewire including a first distal tip. A second guidewire is adapted to be positioned in the receiver, the second guidewire including a second distal tip. A releasable coupling is provided for selectively connecting the first guidewire to the second guidewire such that, in a coupled configuration, the first distal tip and the second distal tip are aligned.

In one embodiment, a first proximal tip of the first guidewire and a second proximal tip of the second guidewire are aligned in a coupled configuration. In this coupled configuration, a second proximal tip of the second guidewire may be aligned with a first proximal tip of the first guidewire, but may be located proximally of the first proximal tip of the first guidewire. In any case, the second guidewire has a second length equal to or greater than a first length of the first guidewire.

In one embodiment, the releasable coupling comprises a magnetic coupling. The receiver comprises a lateral channel or a bore. The releasable coupling may comprise a transverse pin. In any disclosed embodiment, a first diameter of the first guidewire is greater than a second diameter of the second guidewire.

### BRIEF DESCRIPTION OF THE DRAWINGS

The above and further advantages of the present disclosure may be better understood by referring to the following description in conjunction with the accompanying drawings in which:

FIG. 1 is a front perspective, cutaway view of a guidewire assembly according to one aspect of the disclosure.

FIG. 2 is an enlarged, partially cutaway view of the guidewire assembly of FIG. 1.

FIG. 3 is an end view of the guidewire assembly of FIG. 1.

FIG. 4 is an enlarged, partially cutaway view of the guidewire assembly of FIG. 1, in a detached state.

FIGS. 5 and 6 illustrate alternative embodiments of a guidewire assembly.

FIGS. 7 and 8 illustrate further alternative embodiments of a guidewire assembly.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the present inventive concepts.

The invention is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

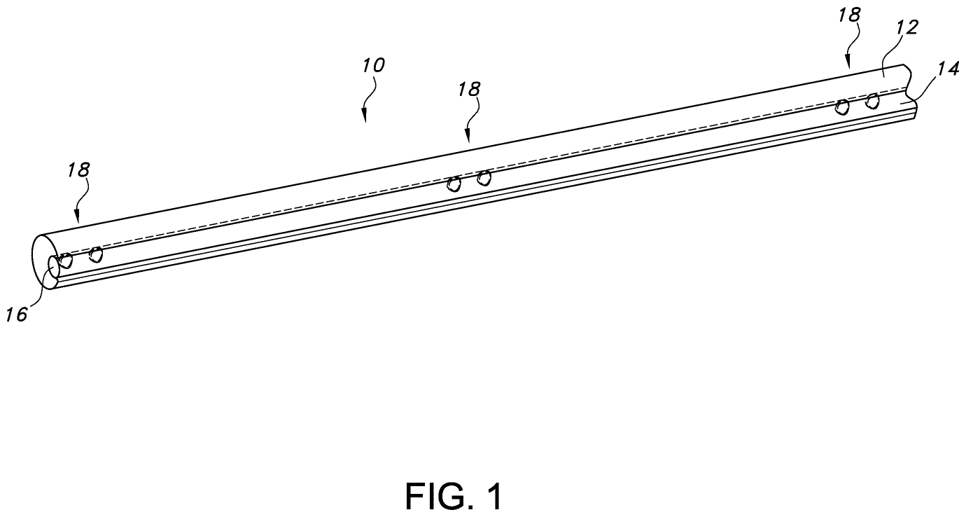

With reference to FIGS. 1-4, a guidewire assembly 10 is shown. The assembly 10 comprises a first or outer guidewire 12 having a larger diameter than a second or inner guidewire 14, and which may have identical lengths (but this is considered optional). Both the guidewire assembly 10 and the first and second guidewires 12, 14 forming it each comprise a thin, elongated structure, typically formed of a flexible, resilient material to as to advance through the vascular and, in particular, a tortuous vessel, as will be further outlined in the following description.

The first guidewire 12 includes a receiver for receiving the second guidewire 14 so as to complete the guidewire assembly 10. In the embodiment of FIGS. 1-4, this receiver comprises a lateral channel 16 by which the second guidewire 14 is received. As can perhaps be best understood from FIG. 3, the channel 16 may have a C-shape when the open end formed by the surface of the guidewire 12 is oriented toward one side (or U-shape, if the open end is oriented vertically), with a rounded inner surface 16a and generally parallel sidewalls 16b. As can be appreciated, forming the channel 16 in this manner also provides the first guidewire 12 with a C-shaped (or U-shaped) cross-section, depending on the relative orientation.

The channel 16 may be sized and shaped to allow for the insertion and removal of the second guidewire 14 in a radial direction R, without interference (which is also known as “peelability”). Specifically, an open end of the channel 16 (that is, a lateral opening extending along the length of the channel) has a dimension D greater than the diameter of the second guidewire 14. The length of the channel 16 may be equal to the length of the guidewire 14, but this is considered optional.

In order to retain the first and second guidewires 12, 14 in an assembled condition, a magnetic coupling 18 may be utilized. In the illustrated embodiment, this magnetic coupling is formed by one or more magnet pairs 18a, 18b having opposite polarities. In one example, a first pair of magnets 18a may be at least partially embedded in the first guidewire 12 along the curved or rounded inner surface 16a, and a corresponding second pair of magnets 18b may be provided in opposition attached along or at least partially embedded in a rear surface of the second guidewire 14. The magnetic coupling 18 may be established by providing spaced magnet pairs 18a, 18b at periodic intervals along the length of the assembly 10, as necessary to ensure that the desired attachment is maintained prior to and during use as an assembly, but the coupling force is not great enough to prevent separation or peeling of the first guidewire 12 and second guidewire 14 when desired.

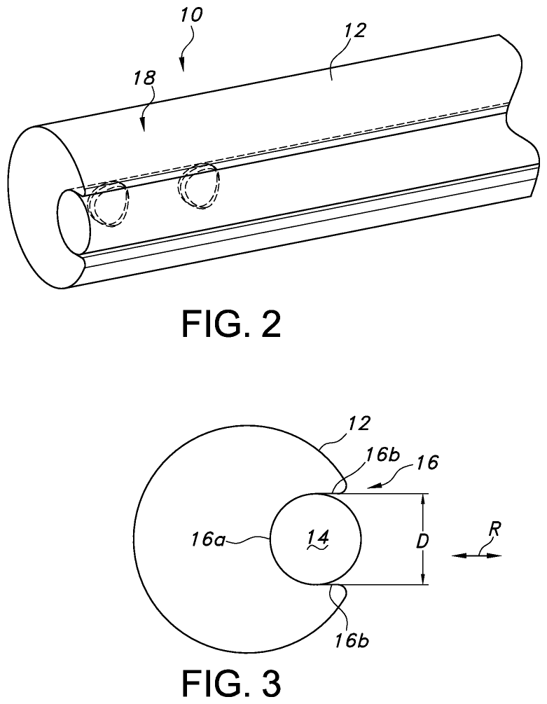

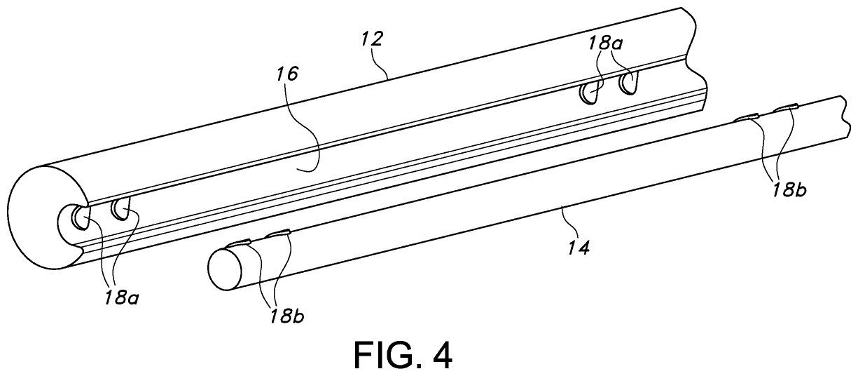

In use, the guidewire assembly 10 may be used as a unit when the larger diameter guidewire 12 is needed for a particular procedure. When a smaller diameter guidewire 14 is needed, the magnetic coupling 18 may be released by peeling the second guidewire 14 from the channel 16. Upon detachment, as shown in FIG. 4, it is ready for independent use, as is guidewire 12. If reattachment is desired, such as for the same or a different procedure, the first and second guidewires 12, 14 may be reunited as shown in FIGS. 1 and 2, with the magnetic coupling 18 being easily reestablished by aligning the magnet pairs 18a, 18b.

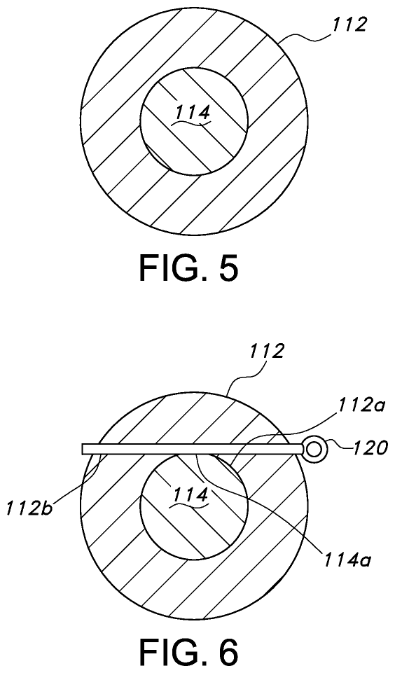

An alternative embodiment of a guidewire assembly 100 is described herein with reference to FIGS. 5 and 6. In this embodiment, the guidewire assembly 100 comprises a first, larger diameter guidewire 112 including a receiver in the form of a closed passage or bore 112a adapted to receive and surround the second, smaller diameter guidewire 114. Hence, the manner of separation of the guidewires 112, 114 is by pulling or sliding one of them (usually, the smaller, inner guidewire 114) in the longitudinal or axial direction, rather than the transverse or radial direction. While the bore 112a is shown as being circular in cross-section and aligned with a center of the guidewire 112, which is also circular in cross-section, it may instead be radially offset in any desired direction (that is, the arrangement need not be symmetrical, as illustrated).

Retention of the assembly 100 may be achieved in this embodiment using a mechanical connection at a proximal end portion, such as by way of a connector or pin 120 extending transversely through the bore 112a via a transverse passage 112b in the first or outer guidewire 112 intersecting with the second guidewire 114, such as by way of a chordal notch 114a. Removal of the pin 120 thus allows for the guidewires 112, 114 to be separated in the axial direction. If reassembly is desired, the pin 120 may be selectively returned to secure the two guidewires once reassembled and the notch 114a aligned with the transverse passage 112b. As can be appreciated, the pin 120 may also be used in the embodiment of FIGS. 1-4 to releasably secure the guidewires 12, 14 via a mechanical connection. Likewise, the magnetic coupling may also be used in the embodiment of FIGS. 4-5.

Selection of the diameters of the guidewires 12, 14, 112, 114 may depend on the requirements of a particular situation. In a common example, the larger guidewire 12, 112 would be an “035” size, meaning approximately 0.035 inches in diameter, and the smaller guidewire 14, 114 would be an “014” or “018” size, meaning from approximately 0.014 inches up to and including 0.018 inches in diameter, respectively, but these are just possible examples.

The guidewires 12, 14, 112, 114 may be fabricated of commonly used materials such as stainless steel, Nitinol and one or more coating or jacketing layers (which may comprise polymer materials). The magnets for forming the magnetic coupling may be rare Earth, such as neodymium, and could be formed by a single magnet attached to one guidewire in the case where the other is formed of a magnetized (e.g., ferromagnetic) material.

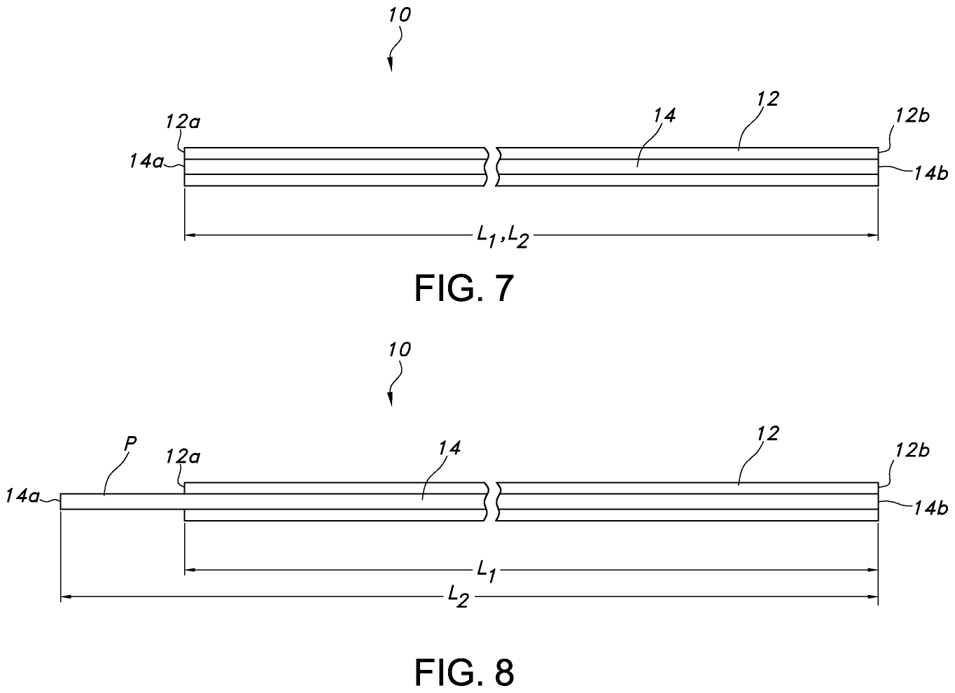

The first and second guidewires 12, 14 (or 112, 114) may be substantially equal in length, as shown in FIG. 7. In other words, length L₁ of the first guidewire 12 and length L₂ of the second guidewire 14 are substantially the same, or L₁=L₂. Consequently, in a coupled configuration, that proximal tips 12a, 14a and distal tips 12b, 14b align in an axial or longitudinal direction.

Alternatively, the second guidewire 14 when nested with the first guidewire 12 may be greater in length (L₂>L₁), such that a proximal end portion P of the second guidewire extends proximally from the first guidewire 12 when the guidewires are nested together and coupled, such as by the magnetic or mechanical coupling. This extending proximal end portion P thus allows for ease of gripping of the second guidewire 14 for purposes of being removed from the first guidewire 12, such as by peeling away or withdrawal in an axial direction. The distal end tips 12b, 14b at the opposite, distal end portions align when the guidewires 12, 14 are in the coupled configuration, but the proximal tips 12a, 14a are spaced apart in an axial or longitudinal direction.

This disclosure may also be considered to relate to a method of performing a medical procedure in a vasculature. The method may include inserting into the vasculature a guidewire assembly formed of a first guidewire releasably coupled to a second guidewire having a smaller diameter than the first guidewire. After the inserting step, the method may involve separating the first guidewire from the second guidewire. In the case of the embodiment of FIGS. 1-4, this may include breaking a magnetic coupling formed among the first guidewire and second guidewire, but could also involve releasing a mechanical coupling formed between the first guidewire and second guidewire. In the FIG. 1-4 embodiment, the separating step may comprise peeling away the second guidewire from a lateral channel in the first guidewire.

Summarizing, this disclosure may be considered to relate to the following items:
1. A detachable guidewire assembly, comprising:
   a first guidewire having a first diameter and including a lateral channel;
   a second guidewire having a second diameter less than the first diameter, the second guidewire adapted for being inserted within and removed from the lateral channel; and
   a releasable coupling for selectively connecting the first guidewire to the second guidewire.
2. The detachable guidewire assembly of item 1, wherein the releasable coupling comprises a magnetic coupling.
3. The detachable guidewire assembly of item 2, wherein the magnetic coupling is formed by a plurality of magnets arranged in pairs spaced along the first guidewire and the second guidewire.
4. The detachable guidewire assembly of any of items 1-3, wherein the releasable coupling comprises a transverse pin.
5. The detachable guidewire assembly of any of items 1-4, wherein the lateral channel includes an open end having a dimension greater than the second diameter.
6. The detachable guidewire assembly of any of items 1-5, wherein the first diameter is from approximately 0.035 inches to 0.039 inches and the second diameter is from approximately 0.014 to 0.018 inches.
7. The detachable guidewire assembly of any of items 1-6, wherein the first guidewire comprises a C-shaped cross-section.
8. A detachable guidewire assembly, comprising:
   a first guidewire having a first diameter;
   a second guidewire having a second diameter less than the first diameter; and
   a magnetic coupling for selectively connecting the first guidewire to the second guidewire.
9. The detachable guidewire assembly of item 8, wherein the first guidewire includes a receiver adapted for receiving the second guidewire.
10. The detachable guidewire assembly of item 9, wherein the receiver comprises a lateral channel or a bore adapted for receiving the second guidewire.
11. The detachable guidewire assembly of item 10, wherein the receiver is the lateral channel and includes an open end having a dimension greater than the second diameter.
12. The detachable guidewire assembly of any of items 8-11, wherein the magnetic coupling is formed by a plurality of magnets arranged in pairs spaced along the first and second guidewire.
13. The detachable guidewire assembly of any of items 8-12, wherein the first diameter is from approximately 0.035 inches to 0.039 inches and the second diameter is from approximately 0.014 to 0.018 inches.
14. The detachable guidewire assembly of any of items 10-13, wherein the first guidewire comprises a C-shaped cross-section.
15. A detachable guidewire assembly, comprising:
   a first guidewire including a receiver;
   a second guidewire adapted to be positioned in the receiver; and
   a releasable coupling within the receiver for selectively connecting the first guidewire to the second guidewire.
16. The detachable guidewire assembly of item 15, wherein the releasable coupling comprises a magnetic coupling.
17. The detachable guidewire assembly of item 16, wherein the magnetic coupling is formed by a plurality of magnets arranged in pairs spaced along the first guidewire and the second guidewire.
18. The detachable guidewire assembly of any of items 15-17, wherein the releasable coupling comprises a transverse pin.
19. The detachable guidewire assembly of any of items 15-18, wherein the receiver includes an open end having a dimension greater than a diameter of the second guidewire.
20. The detachable guidewire assembly of any of items 15-19, wherein a first diameter of the first guidewire is from approximately 0.035 inches to 0.039 inches and a second diameter of the second guidewire is from approximately 0.014 to 0.018 inches.
21. A detachable guidewire assembly, comprising:
   a first guidewire including a receiver;
   a second guidewire adapted to be located in the receiver; and
   a transverse pin for selectively connecting the first guidewire to the second guidewire.
22. The detachable guidewire assembly of item 21, wherein the first guidewire includes a transverse passage for receiving the transverse pin so as to pass through a notch in the second guidewire when located in the receiver.
23. The detachable guidewire assembly of item 21 or item 22, wherein the receiver comprises a bore in the first guidewire.
24. The detachable guidewire assembly of any of items 21-23, wherein a first diameter of the first guidewire is from approximately 0.035 inches to 0.039 inches and a second diameter of the second guidewire is from approximately 0.014 to 0.018 inches.
25. The detachable guidewire assembly of any of items 1-24, wherein the second guidewire has a second length equal to or greater than a first length of the first guidewire.
26. A detachable guidewire assembly, comprising:
   a first guidewire including a receiver, the first guidewire including a first distal tip;
   a second guidewire adapted to be positioned in the receiver, the second guidewire including a second distal tip; and
   a releasable coupling for selectively connecting the first guidewire to the second guidewire such that, in a coupled configuration, the first distal tip and the second distal tip are aligned.
27. The detachable guidewire assembly of item 26, wherein in the coupled configuration, a first proximal tip of the first guidewire and a second proximal tip of the second guidewire are aligned.
28. The detachable guidewire assembly of item 26, wherein in the coupled configuration, a second proximal tip of the second guidewire is located proximally of a first proximal tip of the first guidewire.
29. The detachable guidewire assembly of any of items 26-28 wherein the second guidewire has a second length equal to or greater than a first length of the first guidewire.
30. The detachable guidewire assembly of any of items 26-29, wherein the releasable coupling comprises a magnetic coupling.
31. The detachable guidewire assembly of any of items 26-29, wherein the receiver comprises a lateral channel or a bore.
32. The detachable guidewire assembly of any of items 26-31, wherein the releasable coupling comprises a transverse pin.
33. The detachable guidewire assembly of any of items 26-32, wherein a first diameter of the first guidewire is greater than a second diameter of the second guidewire.
34. A method of performing a medical procedure in a vasculature, comprising:
   inserting into the vasculature a guidewire assembly formed of a first guidewire releasably coupled to a second guidewire having a smaller diameter than the first guidewire; and
   after the inserting step, separating the first guidewire from the second guidewire.
35. The method of item 34, wherein the separating step comprises breaking a magnetic coupling formed among the first guidewire and second guidewire.
36. The method of item 34, wherein the separating step comprises releasing a mechanical coupling formed between the first guidewire and second guidewire.
37. The method of item 34, wherein the separating step comprises peeling away the second guidewire from a lateral channel in the first guidewire or pulling or sliding the second guidewire from a bore in the first guidewire.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

All publications, patents and patent applications mentioned in this specification are herein incorporated in their entirety by reference into the specification, to the same extent as if each individual publication, patent or patent application was specifically and individually indicated to be incorporated herein by reference. In addition, the citation or identification of any reference in this application shall not be construed as an admission that such reference is available as prior art to the present disclosure.

1. A detachable guidewire assembly, comprising:
a first guidewire having a first diameter and including a lateral channel;
a second guidewire having a second diameter less than the first diameter, the second guidewire adapted to be inserted within and removed from the lateral channel; and
a releasable coupling for selectively connecting the first guidewire to the second guidewire.

2. The detachable guidewire assembly of claim 1, wherein the releasable coupling comprises a magnetic coupling.

3. The detachable guidewire assembly of claim 2, wherein the magnetic coupling is formed by a plurality of magnets arranged in pairs spaced along the first guidewire and the second guidewire.

4. The detachable guidewire assembly of claim 1, wherein the releasable coupling comprises a pin extending transversely across the first guidewire and second guidewire.

5. The detachable guidewire assembly of claim 1, wherein the lateral channel includes a lateral open end having a dimension greater than the second diameter.

6. (canceled)

7. The detachable guidewire assembly of claim 1, wherein the first guidewire comprises a C-shaped cross-section.

8. The detachable guidewire assembly of claim 1, wherein the second guidewire has a second length equal to or greater than a first length of the first guidewire.

9. The detachable guidewire assembly of claim 1, wherein the second guidewire is longer than the first guidewire.

10. A detachable guidewire assembly, comprising:
a first guidewire having a first diameter;
a second guidewire having a second diameter less than the first diameter; and
a magnetic coupling for selectively connecting the first guidewire to the second guidewire.

11. The detachable guidewire assembly of claim 10, wherein the first guidewire includes a receiver adapted for receiving the second guidewire.

12. The detachable guidewire assembly of claim 11, wherein the receiver comprises a lateral channel adapted for receiving the second guidewire.

13. The detachable guidewire assembly of claim 11, wherein the lateral channel includes a laterally open end having a dimension greater than the second diameter.

14. The detachable guidewire assembly of claim 10, wherein the magnetic coupling is formed by a plurality of magnets arranged in pairs spaced along the first and second guidewire.

15. (canceled)

16. The detachable guidewire assembly of claim 10, wherein the first guidewire comprises a C-shaped cross-section.

17. The detachable guidewire assembly of claim 10, wherein the second guidewire has a second length equal to or greater than a first length of the first guidewire.

18. A detachable guidewire assembly, comprising:
a first guidewire including a receiver;
a second guidewire adapted to be positioned in the receiver; and
a releasable coupling within the receiver for selectively connecting the first guidewire to the second guidewire.

19. The detachable guidewire assembly of claim 18, wherein the releasable coupling comprises a magnetic coupling.

20. The detachable guidewire assembly of claim 19, wherein the magnetic coupling is formed by a plurality of magnets arranged in pairs spaced along the first guidewire and the second guidewire.

21. The detachable guidewire assembly of claim 18, wherein the releasable coupling comprises a pin extending transversely across the first guidewire and the second guidewire.

22. The detachable guidewire assembly of claim 18, wherein the receiver includes an open end having a dimension greater than a diameter of the second guidewire.

23.-40. (canceled)
